(19) United States
(12) Patent Application Publication
(10) Pub. No.: US 2012/0100551 A1
(43) Pub. Date: Apr. 26, 2012

(54) NUCLEIC ACID AMPLIFICATION REACTION DEVICE, SUBSTRATE USED FOR NUCLEIC ACID AMPLIFICATION REACTION DEVICE, AND NUCLEIC ACID AMPLIFICATION REACTION METHOD

(75) Inventors: KENSUKE KOJIMA, KANAGAWA (JP); JUNJI KAJIHARA, TOKYO (JP)

(73) Assignee: SONY CORPORATION, Tokyo (JP)

(21) Appl. No.: 13/267,980

(22) Filed: Oct. 7, 2011

(30) Foreign Application Priority Data
Oct. 22, 2010 (JP) 2010-237174

(51) Int. Cl. C12Q 1/68; G02B 5/08; C12M 1/42

(52) U.S. Cl. 435/6.12; 435/287.2; 359/838

(57) ABSTRACT

Disclosed herein is a nucleic acid amplification reaction device including: a reaction area configured to serve as a reaction field of a nucleic acid amplification reaction; an irradiating unit configured to irradiate light to the reaction area; and a light detecting unit configured to detect the amount of reflected light, wherein a reflective component that reflects side light generated in the reaction area due to light irradiation from the irradiating unit and guides the light to the light detecting unit is disposed.

### CROSS REFERENCES TO RELATED APPLICATIONS

The present application claims priority to Japanese Priority Patent Application JP 2010-237174 filed in the Japan Patent Office on Oct. 22, 2010, the entire content of which is hereby incorporated by reference.

### BACKGROUND

The present disclosure relates to nucleic acid amplification reaction devices, substrates used for nucleic acid amplification reaction devices, and nucleic acid amplification reaction methods, and particularly to a nucleic acid amplification reaction device including a reflective component to reflect side light in a reaction area serving as the reaction field of a nucleic acid amplification reaction.

Techniques to amplify a specific nucleic acid, such as polymerase chain reaction (PCR), are applied in various fields in the biotechnology. In general, the nucleic acid amplification reaction such as the PCR requires a step of checking whether or not the target nucleic acid is specifically amplified. For example, there is a method in which the check is performed by subjecting the reaction liquid used for a nucleic acid amplification reaction such as the PCR to gel electrophoresis by use of a gel of e.g. polyimide and thereafter staining DNA fragments obtained by the PCR amplification.

Furthermore, e.g. the following related-art methods are also used as a method for checking nucleic acid amplification in a nucleic acid amplification reaction: a method of checking amplification by measuring the turbidity of the reaction liquid used for a nucleic acid amplification reaction; a method of using a microarray including a probe specifically coupled to the nucleic acid as the amplification subject; and real-time PCR in which amplification is checked in real time by using a fluorescently labeled probe coupled to a double-stranded DNA or a fluorescently labeled probe specifically coupled to the target PCR product.

The nucleic acid amplification reaction such as the PCR is used also for analysis of e.g. a single nucleotide polymorphism (SNP) and the above-described methods for checking nucleic acid amplification are used.

There has been proposed an analysis method in which a primer for a wild type and one or two kinds of primers for a variant type are made to simultaneously or separately act on a chromosome or a fragment thereof including the SNP site as the analysis subject together with a DNA polymerase to examine whether or not extension based on the primer is present, and electrophoresis is used as the method for checking the amplified nucleic acid.

Furthermore, there has been proposed an SNP analysis method in which the target sequence part is amplified by using two kinds of specific primers for the reference sequence including the SNP site and for a variant sequence and a universal primer, and whether or not the amplification product is present is checked by subjecting the obtained reaction to electrophoresis. However, the electrophoresis takes too long a time and involves the influence of contamination.

On the other hand, there has been proposed a method in which typing is performed by amplifying a nucleic acid including the SNP site by using the analysis-subject genome DNA and plural pairs of primers. This typing is performed by e.g. hybridization with use of a labeled probe or the like for the obtained amplification product.

If the SNP analysis can be performed rapidly and easily, e.g. personalized medicine to diagnose the optimum treatment method, medication method, and so forth at the bedside of a patient or the like is enabled and a competent POC (Point Of Care) technique is established. For this purpose, a method for checking nucleic acid amplification after nucleic acid amplification reaction more rapidly and easily is desired.

As a method for detecting a nucleic acid with use of a hybridization probe labeled with a fluorescent substance, e.g. a nucleic acid quantification method (real-time PCR) and a method for detecting a variant such as a single nucleotide polymorphism (SNP) (melting curve analysis) are known.

A probe whose fluorescence intensity changes between the hybridized state (including also a state of being cut after being hybridized) and the free state is used as the fluorescently labeled hybridization probe used in these methods, and detection is performed by measuring this change. A representative thereof is a probe utilizing fluorescence resonance energy transfer (FRET) and TaqMan (trademark) probe and molecular beacon are known as examples of such a probe.

In the probe utilizing the FRET, two kinds of fluorescent dyes, reporter dye and quencher dye, need to be used. Thus, the design of the probe is complicated.

So, there is known a nucleic acid probe that utilizes a phenomenon that the light emission of a fluorescent dye decreases when a nucleic acid probe labeled with the fluorescent dye is hybridized with the target nucleic acid and uses one kind of fluorescent dye for the purpose of quantifying a nucleic acid more easily (refer to Japanese Patent Laid-open No. 2005-261354 and Japanese Patent Laid-open No. 2002-119291). Furthermore, regarding a probe labeled with Alexa flour (registered trademark) 350, 488, 568; Pacific Blue (registered trademark), and Cy3, it is also known that the light emission of a fluorescent dye increases in some cases when the labeled probe is hybridized (refer to Marras SAE, Kramer FR, and Tyagi S. (2002), Nucleic Acids Research, 30, e122).

The following methods are known as a detecting method in which an electrophoresis gel, a support body such as a film, and a labeled substance are not used.

For example, there are known a method in which polarized light is made to pass through a nucleic acid amplification reaction liquid and the optical rotation and the circular dichroism are measured (refer to Japanese Patent Laid-open No. 2002-186481) and a method of sensing change in the polarized light component of the extended amplification product (refer to Japanese Patent Laid-open No. 2002-171997, Japanese Patent Laid-open No. 2002-171998, and Japanese Patent Laid-open No. 2002-171999).

For example, there is known a method of observing precipitation of an insoluble substance due to the pyrophosphoric acid generated in association with an amplification reaction and magnesium (refer to International Patent Publication WO 01/83817 brochure). Furthermore, there is known a method in which the pyrophosphoric acid as the amplification product is treated with an enzyme reaction reagent containing an oxidase and electron transfer occurring when the oxidase acts is amplified under the existence of an electrochemically active intercalater to be electrochemically detected as a current (refer to Japanese Patent Laid-open No. 2003-299 and Japanese Patent Laid-open No. 2003-47500). In addition, there is known a method of detecting whether or not nucleic acid amplification is present by sensing change in the amount of metal ions in the reaction liquid based on the difference in the coupling capability between dNTP and the pyrophosphoric acid in a nucleic acid amplification reaction by a metal indicator (Japanese Patent Laid-open No. 2004-283161).

### SUMMARY

Although the above-described detecting methods are frequently used, many disadvantages are also found in detection of nucleic acid amplification. For example, in the case of the nucleic acid probe using a fluorescent dye, many probes have small difference between the excitation wavelength and the fluorescence wavelength, i.e. small Stokes shift, although fluorescence sensitization when the probe is intercalated into a double-stranded nucleic acid in nucleic acid detection is large. Therefore, there are also many disadvantages in terms of crosstalk and the gain. Furthermore, the detection based on precipitation of the magnesium pyrophosphate has an aspect that the signal recognition performance and the appeal power are somewhat poor, although it is extremely easy and practical.

Therefore, in detection (reaction) of nucleic acid amplification, enhancement in the detection sensitivity with an easy-to-use configuration is required.

There is a desire for a technique to provide a nucleic acid amplification reaction device that is easy to use and allows achievement of high detection sensitivity, a substrate used for a nucleic acid amplification reaction device, and a nucleic acid amplification reaction method.

According to an embodiment of the present disclosure, there is provided a nucleic acid amplification reaction device including a reaction area configured to serve as a reaction field of a nucleic acid amplification reaction, an irradiating unit configured to irradiate light to the reaction area, and a light detecting unit configured to detect the amount of reflected light. In the nucleic acid amplification reaction device, a reflective component that reflects side light generated in the reaction area due to light irradiation from the irradiating unit and guides the light to the light detecting unit is disposed.

According to another embodiment of the present disclosure, there is provided a substrate including a reflective component configured to reflect side light from a reaction area serving as a reaction field of a nucleic acid amplification reaction.

According to another embodiment of the present disclosure, there is provided a nucleic acid amplification reaction method including guiding side light that is generated due to light irradiation and is from a reaction area serving as a reaction field of a nucleic acid amplification reaction into a light output surface direction and/or a light incident surface direction by a reflective component disposed around the reaction area, and detecting the amount of guided light by a light detector.

The embodiments of the present disclosure provide a nucleic acid amplification reaction device, a substrate, and a nucleic acid amplification reaction method that are easy to use and allow achievement of higher detection sensitivity.

Additional features and advantages are described herein, and will be apparent from the following Detailed Description and the figures.

### BRIEF DESCRIPTION OF THE FIGURES

FIG. 1 is a conceptual diagram of a nucleic acid amplification reaction device according to an embodiment of the present disclosure (first embodiment);

FIGS. 2A and 2B show an example of a section of an area around a reaction area along the light incident surface direction-light output surface direction in a substrate according to an embodiment of the present disclosure, and the optical path therein;

FIG. 3 is a perspective view of the area around the reaction area in the substrate according to the embodiment of the present disclosure;

FIGS. 4A to 4C show examples of the substrate around the reaction area according to the embodiment of the present disclosure;

FIGS. 5A to 5G simply show manufacturing procedures of the substrate according to the embodiment of the present disclosure;

FIGS. 6A to 6F show examples of a method for fabricating a resin mold used for manufacturing of the substrate according to the embodiment of the present disclosure; and

FIG. 7 is a conceptual diagram of a nucleic acid amplification reaction device according to an embodiment of the present disclosure (second embodiment).

### DETAILED DESCRIPTION

Embodiments of the present application will be described below in detail with reference to the drawings.

1. Nucleic Acid Amplification Reaction Device (First Embodiment)

(1) Reaction Area

(1-a) Reflective Component

(1-b) Sidewall Part

(1-c) Phosphor Component

(2) Substrate

(2-a) Method for Manufacturing Substrate

(3) Nucleic Acid Amplification Reaction

(3-a) Method for Detecting Nucleic Acid Amplification (Product)

(4) Irradiating Unit

(5) Temperature Control Unit

(6) Light Detecting Unit

2. Operation of Nucleic Acid Amplification Reaction Device (First Embodiment)

(1) Detection of Light Component Derived from Turbidity Substance in Nucleic Acid Amplification Reaction

(1-a) When Phosphor Component. Is Not Present in Substrate

(1-b) When Phosphor Component Is Present in Substrate

(2) Detection of Light Component Derived from Fluorescent. Substance in Nucleic Acid Amplification Reaction

(2-a) When Phosphor Component Is Not Present in Substrate

(2-b) When Phosphor Component Is Present in Substrate

3. Nucleic Acid Amplification Reaction Device (Second Embodiment)

4. Operation of Nucleic Acid Amplification Reaction Device (Second Embodiment)

5. Modification Examples

(1) Operation of RT-LAMP Device

(2) Operation of RT-PCR Device

### 1. Nucleic Acid Amplification Reaction Device

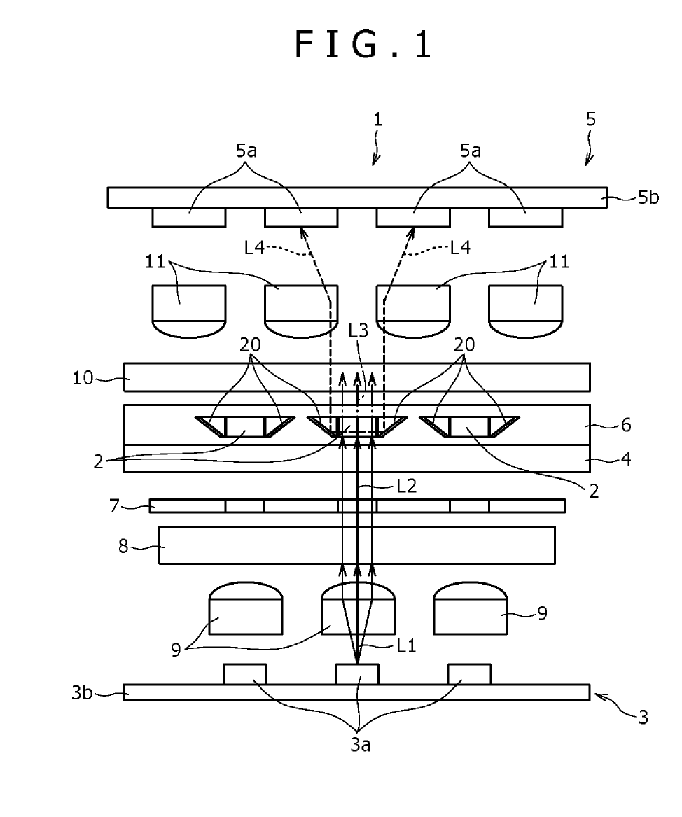

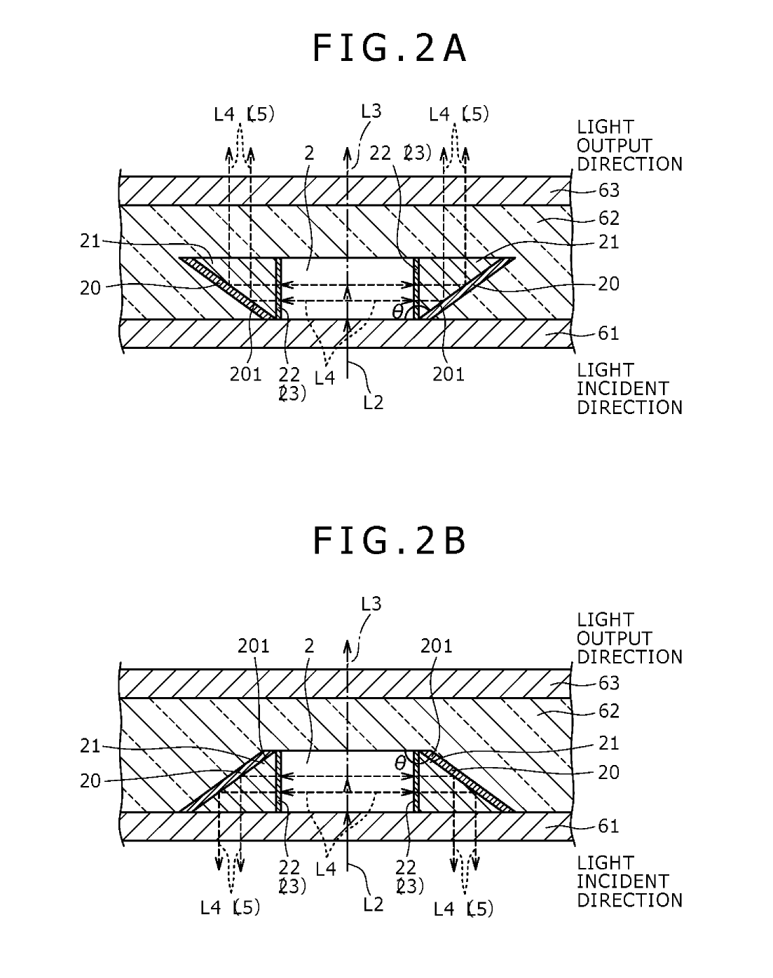

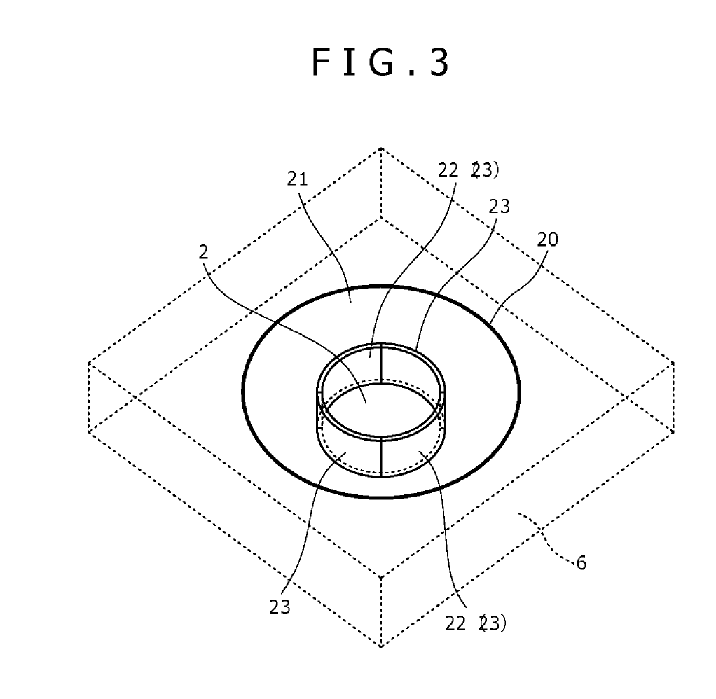

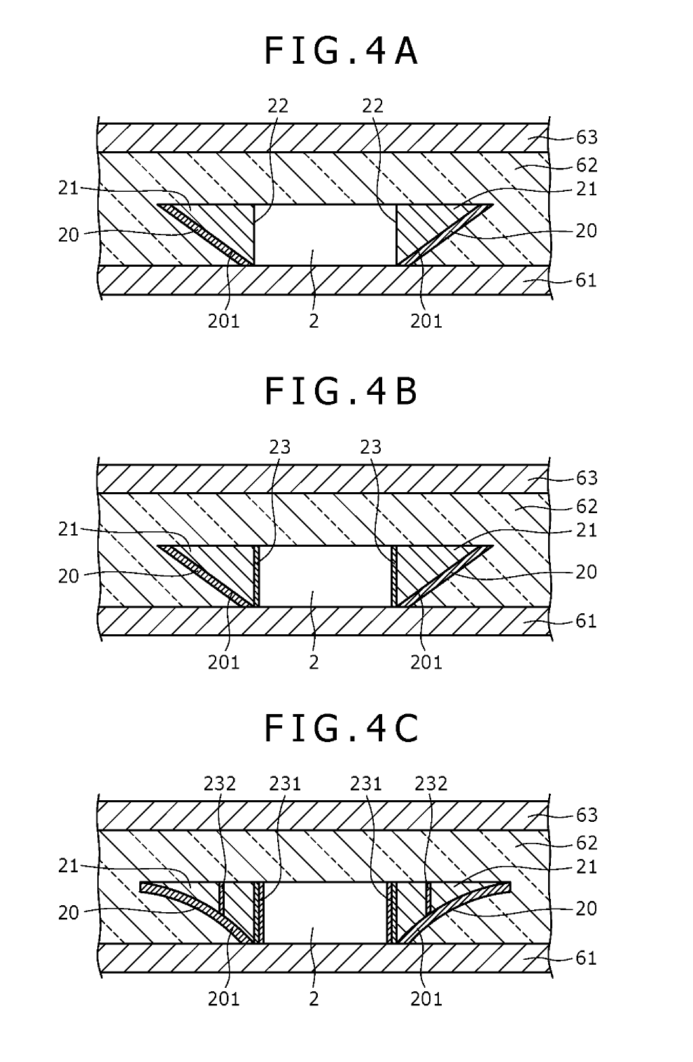

FIG. 1 is a conceptual diagram of a nucleic acid amplification reaction device 1 according to an embodiment of the present disclosure (first embodiment). FIGS. 2A and 2B show a sectional view of an area around a reaction area along the light incident surface direction-light output surface direction in a substrate according to an embodiment of the present disclosure, and an example of the optical path therein. FIG. 3 is a perspective view of the area around the reaction area in the substrate according to the embodiment of the present disclosure. FIGS. 4A to 4C show examples of the substrate around the reaction area according to the embodiment of the present disclosure.

In the drawings described below, the device configuration and so forth is shown in a simplified manner and so forth, for convenience of description.

The nucleic acid amplification reaction device according to the embodiment of the present disclosure (first embodiment) shown in FIG. 1 includes a reaction area 2, an irradiating unit 3, and a light detecting unit 5 for controlling a nucleic acid amplification reaction to amplify and quantify a nucleic acid, and is provided with a temperature control unit arbitrarily.

In the nucleic acid amplification reaction device 1 of the embodiment of the present disclosure, a temperature control unit 4 and the reaction area 2 (substrate 6) that is detachable are disposed between the irradiating unit 3 and the light detecting unit 5. Furthermore, a pinhole 7, an excitation filter 8, and a collecting lens 9 may be disposed between the reaction area 2 and the irradiating unit 3 arbitrarily in order to adjust the amount of light, the light component, and so forth. In addition, a fluorescent filter 10 and a collecting lens 11 may be disposed between the reaction area 2 and the light detecting unit 5 arbitrarily in order to adjust the amount of light, the light component, and so forth. It is preferable that the nucleic acid amplification reaction device 1 of the embodiment of the present disclosure be provided with a controller (not shown) to control respective kinds of operation relating to the device of the embodiment of the present disclosure (e.g. light control, temperature control, nucleic acid amplification reaction, light detection control, calculation of the amount of detected light, and monitoring).

The respective configurations will be described in detail below.

(1) Reaction Area

The reaction area 2 is an area serving as the reaction field of an amplification reaction of a nucleic acid and is disposed at such a position that it can be irradiated with light from the irradiating unit 3 (see FIGS. 1, 2A, 2B, and 7). A nucleic acid amplification product is generated in this reaction area 2 in association with the progression of an amplification reaction, and light is generated toward the lateral side of the reaction area 2 when this nucleic acid amplification product is irradiated with light from the irradiating unit 3. A reflective component 20 is so disposed that this side light is guided to the light detecting unit 5 (see FIGS. 1, 2A, 2B, and 7).

The shape of the reaction area 2 is not particularly limited as long as an area serving as the reaction field of an amplification reaction of a nucleic acid is provided inside. Examples of the shape include cylindrical shape, conical frustum shape, pyramidal frustum shape (e.g. rectangular frustum shape), and cubic shape.

(1-a) Reflective Component

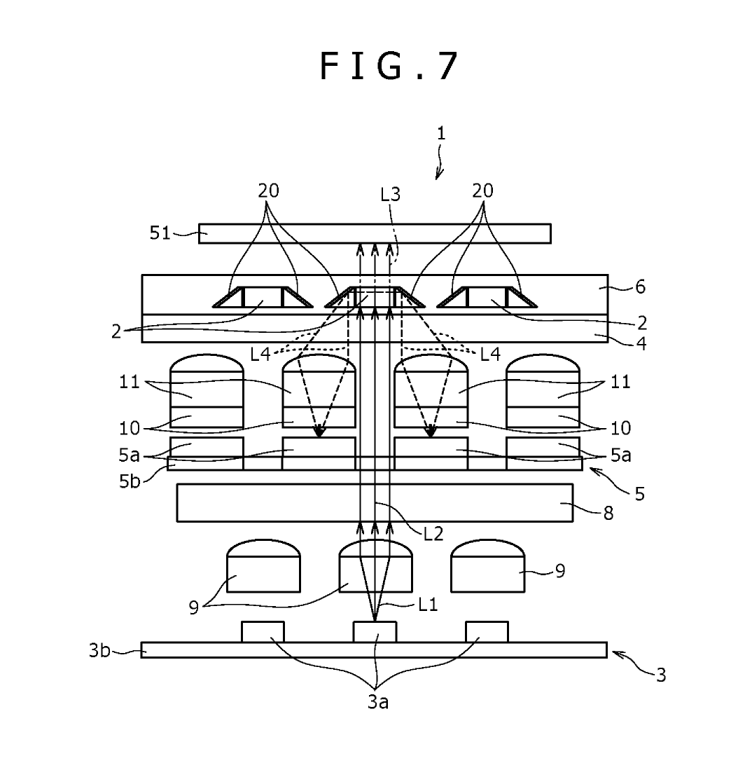

The reflective component 20 is not particularly limited as long as it is so disposed as to reflect the side light from the reaction area 2 and guide the light to the light detecting unit 5 finally (see FIGS. 1 and 7). It is preferable that, as shown in FIGS. 2A and 2B, the reflective component 20 (reflective surface 201) be so disposed around the reaction area 2 as to guide the side light from the reaction area into the light output surface direction and/or the light incident surface direction for example.

In this case, the reflection direction of the side light may be adjusted by utilizing plural reflective components 20 (reflective surfaces 201) (not shown). For example, the side light may be reflected substantially horizontally by one reflective surface and subsequently the side light may be reflected into the light output surface direction and/or the light incident surface direction by another reflective surface.

The reflective surface 201 of the reflective component 20 when the reflective component 20 is cut along the light incident surface direction-light output surface direction may be any surface as long as it is an inclined surface capable of reflecting the side light. Examples of the inclined surface include flat surface, curve, and flat surface partially having a curve (see e.g. FIGS. 2A, 2B and 4A to 4C). As the obtuse angle (θ) formed by this reflective surface 201 and the surface of the reaction area 2 intersecting it, an angle θ in the range of 90 degrees<θ≦150 degrees is preferable (see FIGS. 2A and 2B).

Furthermore, the three-dimensional shape of the reflective component 20 may be any shape as long as the reflective component 20 can efficiently guide the side light into the light output surface direction and/or the light incident surface direction (see FIGS. 3 and 4A to 4C). Examples of the three-dimensional shape include trumpet shape, conical frustum shape, and pyramidal frustum shape.

The whole surface (all) of the three-dimensional shape may be used for reflection of the side light, or part (one block) of the whole surface of the reflective component 20 may be used. Alternatively, the three-dimensional shape may be divided into plural blocks and each block may be used. As one example, the output direction of the side light may be changed on each divided block basis. For example, the blocks may be disposed in such a manner that one block can output light in the light output surface direction and the other blocks can output light in the light incident surface direction. Such a block may be disposed singularly or plurally around the reaction area arbitrarily.

The material for light reflection by the reflective component 20 (reflective surface 201) may be any material as long as the material provides high reflectance of the side light. Examples of the material include one or more kinds of metal film materials selected from silver, gold, aluminum, rhodium, etc. Among them, silver and a material composed mainly of silver are preferable. By ion sputtering with use of this material, a single-layer or multiple-layer metal film to reflect the side light can be formed as the reflective component 20 (reflective surface 201). It is enough that the thickness of the metal film is, but not particularly limited to, about 30 to 200 nm and the thickness per one layer of the metal film is about 30 to 70 nm.

In the related-art detecting system utilizing transmitted light, particularly in a turbidity detecting system, the S/N ratio is low and sufficient determination is difficult. In contrast, providing the above-described reflective component makes it easy to extract the side light from the reaction area and thereby enhances the projected surface area. Particularly in the turbidity detecting system, almost no side scattering is present at the measurement initial stage, at which the amount of nucleic acid amplification product (scattering object) is small, and therefore the sufficiently-high S/N ratio can be ensured from the measurement initial stage if this side scattered light is used as the basis. Thus, the determination is also easy from the measurement initial stage and therefore the detection sensitivity can be enhanced although the configuration is simple.

As an advantage achieved by employing the above-described reflective component (reflective surface) (reflective technique), the light detecting unit 5 (light receiver) can be disposed on the side of the light incident surface or the light output surface arbitrarily. Therefore, in flexibility of disposing of the optical system and flexibility of the mounting form of the optical system, employing the above-described reflective component is advantageous in terms of the spatial design.

(1-b) Sidewall Part

A sidewall part 21 is provided between the reaction area 2 and the reflective component 20, and this sidewall part 21 is in contact with the reaction area 2 at a sidewall 22 (see FIGS. 2A to 4C). The sidewall part 21 (sidewall 22) may be divided into plural blocks (see FIG. 3). The sidewall part 21 or each block thereof is formed of a material that transmits or blocks the side light from the reaction area 2 or a material that transmits the necessary light component depending on the purpose.

In order for the side light to pass, the sidewall part 21 (e.g. part between the sidewall 22 and the reflective component 20) or part thereof may be a space or this space may be filled with a plastic material (e.g. material having no specific wavelength selectivity). The material having no wavelength selectivity may be any material as long as at least scattered light and fluorescence are transmitted through the material. Examples of the material include exemplified materials having optical transparency to be described later.

It is preferable that such a plastic material or the like containing a phosphor substance and so forth that reflected light and an unnecessary light component are reduced and the side light becomes alight component having the desired specific wavelength (fluorescence or the like) when the side light is transmitted through the sidewall part 21 (sidewall 22) be used for the sidewall part 21 or part thereof (block).

Furthermore, for part (block) of the sidewall part 21, a material utilizing a substance that blocks (absorbs) light or a plastic material or the like containing such a substance may be used so that unnecessary side light having an influence in light detection may be blocked.

(1-c) Phosphor Component

It is preferable to provide one or plural phosphor components 23 between the reaction area 2 and the reflective component 20. In this case, the sidewall part 21 (sidewall 22) containing the above-described phosphor material may be used as the phosphor component 23 in order to turn the side light to a light component having the desired specific wavelength.

Using the detachable reaction area 2 (substrate 6) having the phosphor component 23 makes it possible to easily extract a light component having the desired specific wavelength (e.g. fluorescence) depending on the method for detecting the nucleic acid amplification product. Moreover, reducing reflected light and an unnecessary light component (e.g. stray light of scattered light) is also permitted. The detection sensitivity is also enhanced although the configuration is easily obtained at low cost in the above-described manner.

For example, the phosphor in the phosphor component is excited and the phosphor component emits fluorescence due to side scattered light of a precipitated substance of the pyrophosphoric acid generated in nucleic acid amplification and a metallic salt. Thus, the fluorescence component can be measured without utilizing a fluorescent substance (fluorescent probe) of a nucleic acid amplification reaction solution. Furthermore, in monitoring, the basis with an initial value 0% is easily set. Thus, there is also an advantage that monitoring of the initial stage is easily performed for the user. In addition, if fluorescence is obtained by side scattered light, it is also possible to apply a filter (e.g. fluorescent (wavelength selective transmissive) filter for noise removal) to the light detecting unit (light receiver). Therefore, enhancing the S/N ratio with respect to the incident light is also enabled.

The phosphor component may be formed as plural layers in the sidewall part 21 (see e.g. phosphor components 231 and 232 in FIG. 4C).

By providing the plural phosphor layers, removing an unnecessary light component in advance is also permitted. Furthermore, the number of filters to remove noise in the device can be reduced. Therefore, the detection sensitivity is enhanced and size slimming of the device itself is also enabled. Furthermore, for example the following effect is achieved by forming plural different layers at certain intervals as shown in FIG. 4C. Specifically, after side scattered light from the reaction area is transmitted through the phosphor component 231 and becomes a fluorescence component, part of the fluorescence component is guided to the light detecting unit 5 by the reflective component. The remaining fluorescence component further passes through the phosphor component 232 and becomes a different fluorescence component. Thereafter, the fluorescence component is guided to the light detecting unit 5 by the reflective component. That is, it is also possible to guide each of different fluorescence components to the light detecting unit 5. Thus, simultaneously obtaining another different piece of information from one sample is also permitted and therefore enhancement in the work efficiency is also enabled. In addition, the number of fluorescent (wavelength selective transmissive) filters in the device can be reduced and therefore size reduction of the device itself is also permitted.

As the material of the phosphor used for the phosphor component, a publicly-known phosphor material may be used depending on the desired fluorescence component (about 300 to 750 nm). As the phosphor material, either an organic phosphor or an inorganic phosphor may be employed. However, an inorganic phosphor is preferable because the cost can be easily lowered and the desired wavelength selection can be easily achieved. Various kinds of inorganic phosphors and organic phosphors will be exemplified below. However, the phosphor material is not limited thereto.

The following materials can be used as the inorganic phosphor material. Any of them may be used solely or two or more kinds of them may be used in combination arbitrarily.

A phosphor composed of sialon (Si—Al—O—N) as the base, particularly a fluorescent material that is composed mainly of α-sialon activated by Eu and is obtained by adding an element such as Ca, Y, or Mg to this α-sialon, is cited (refer to e.g. Japanese Patent Laid-open No. 2009-108223). In addition, a fluorescent material composed of β-sialon having a different structure as the base, an inorganic compound having the same crystalline structure as that of the CaSiAlN₃ crystal, and a fluorescent material having the same crystalline structure as that of A₂Si₅N₈ are also cited. These fluorescent materials have an advantage that white is easily obtained by emitting red and green with a blue LED (light-emitting diode) used as the light source.

Oxide phosphor materials composed of a garnet-based Y₃Al₅O₁₂ as the base are cited. For example, a fluorescent material represented by (Rel-rSmr)₃(All-sGas)₅O₁₂: Ce (0≦r<1, 0≦s≦1, Re is at least one kind of element selected from Y and Gd) as a general expression is cited (refer to e.g. Japanese Patent. Laid-open No. 2009-135545). In addition, a green-series phosphor based on an alkaline earth metal aluminate (general expression: (Cal-a, Ma)O.αAl₂O₃.βCe₂O₃.Tb₂O₃ (M is at least one kind of element selected from Mg, Sr, Ba, and Zn, 0≦a=0.9, 0.5≦α≦5.0, 0.015≦β≦0.40, 0.015≦g≦0.42) and so forth) is cited.

Development of a fluorescent layer based on a rare earth complex and a nematic liquid crystal matrix, a halophosphate phosphor (general expression: (Ml-u-vEuuMnv).mX₂.n(PO₄)₆ (0<u/v<100, 1>u+v, 0<m<10, 0<n<10, 1>10n), M=Mg, Ca, Sr, Ba, X═F, Cl, Br, I), and an alkaline earth silicate phosphor ((Sra, Bab, Caz, Euw)₂SiO₄) are cited (refer to e.g. Japanese Patent Laid-open No. 2005-307035).

A Ca—Al—Si—O—N-based material doped with an Eu ion and oxynitride glass are cited (refer to e.g. Japanese Patent Laid-open No. 2008-227550). In addition, an oxynitride-based fluorescent material, a phosphor obtained by adding a group-V element to a phosphor based on the garnet structure, and red-added yellow phosphor and yellow-green phosphor obtained by adding Eu as an activator agent to an oxide of e.g. Ga, Al, or In and sulfurizing part of the oxide are cited.

Sialon phosphors for a white LED such as yellow “α-sialon” and green “β-sialon” are cited (refer to e.g. Japanese Patent Laid-open No. 2010-116564). A characteristic of the β-sialon is that change in the luminance and the color with respect to temperature rise is smaller compared with a silicate-based green phosphor.

A light conversion material composed of a solidified body formed through continuous, three-dimensional intertwining of different metal oxides (e.g. Al₂O₃ and Y₃Al₅O₁₂) with each other is cited (refer to e.g. Japanese Patent Laid-open No. 2006-173433).

A composite material obtained by precipitation of a YAG crystal in amorphous YAG is cited (refer to e.g. Japanese Patent Laid-open No. 2008-231218).

A semiconductor nanocrystal of CdS or the like and a composite body of a nanocrystal and a metal oxide are cited (refer to e.g. Japanese Patent Laid-open No. 2010-114079). A material obtained by dispersing a semiconductor nanocrystal of ZnS or the like in a polymer matrix is cited (refer to e.g. JP-T-2010-528118).

A dielectric phosphor powder obtained by mixing a dielectric particle that does not absorb LED light of blue and so forth (particle with a wide band gap, AlN, air bubble, or the like) and a fluorescent (phosphorescent) material is cited (refer to e.g. Japanese Patent Laid-open No. 2002-261328).

Examples of the organic phosphor material include the following molecular structure low-molecular series, metal complexes, polymer series, π-conjugated polymer materials, σ-conjugated polymer materials, low-molecular-dye-containing polymer-based materials, and dopants. Any of them may be used solely or two or more kinds of them may be used in combination arbitrarily.

Examples of the molecular structure low-molecular series include distyrylbiphenyl-based blue luminescent material, dimesitylboryl-group-coupled amorphous luminescent material, stilbene-based conjugated dendrimer luminescent material, dipyridyl dicyanobenzene luminescent material, methyl-substituted benzoxazole-based fluorescence and phosphorescence emitting material, distyryl-based red luminescent material, heat-resistant carbazole-based green luminescent material, dibenzochrysene-based blue-green luminescent material, arylamine-based luminescent material, pyrene-substituted oligothiophene-based luminescent, material, divinylphenyl-coupled triphenylene-based luminescent material, perylene-based red luminescent material, PPV oligomer-based luminescent material, (carbazole-cyanoterephthalylidene)-based luminescent material, arylethynyl benzene-based blue fluorescence emitting material, quinquepyridine-based luminescent material, fluorene-based star-shape luminescent material, thiophene-based amorphous green-blue luminescent material, low-molar-mass liquid-crystalline luminescent material, (acetonitrile-triphenyleneamine)-based red luminescent dye, bithiazole-based luminescent material, (carbazole-naphthalimido)-based luminescent dye, sexiphenyl-based blue luminescent material, and dimesitylboryl anthracene-based luminescent material.

Examples of the metal complex include oxadiazole-beryllium blue luminescent complex, europium-based phosphorescence emitting complex, heat-resistant lithium-based blue luminescent complex, phosphorescence emitting phosphine-gold complex, terbium-based luminescent complex, thiophene-aluminum yellow luminescent complex, zinc-based yellow-green luminescent complex, amorphous aluminum-based green luminescent complex, boron-based luminescent complex, terbium-substituted europium-based luminescent complex, magnesium-based luminescent complex, phosphorescence emitting lanthanide-based near-infrared emitting complex, ruthenium-based luminescent complex, and copper-based phosphorescence emitting complex.

Examples of the polymer series include oligophenylenevinylene tetramer luminescent material.

Examples of the π-conjugated polymer material include liquid-crystalline fluorene-based blue polarized light emitting polymer, binaphthalene-containing luminescent polymer, disilanyleneoligothienylene-based luminescent polymer, (fluorene-carbazole)-based blue luminescent copolymer, (dicyanophenylenevinylene-PPV)-based luminescent copolymer, silicon blue luminescent copolymer, conjugated chromophore group-containing luminescent polymer, oxadiazole-based luminescent polymer, PPV-based luminescent polymer, (thienylene-phenylene)-based luminescent copolymer, liquid-crystalline chiral-substituted fluorene-based blue luminescent polymer, spirofluorene-based blue luminescent polymer, thermally-stable diethylbenzene-based luminescent polymer, (binaphthyl-fluorene)-based blue luminescent copolymer, porphyrin-group graft PPV-based luminescent polymer, liquid-crystalline dioctylfluorene-based luminescent polymer, ethylene oxide group-added thiophene-based luminescent polymer, oligothiophene-based luminescent polymer, PPV-based blue luminescent polymer, thermally-stable acetylene-based luminescent polymer, (oxadiazole-carbazole-naphthalimide)-based luminescent copolymer, (vinyl pyridine)-based gel luminescent polymer, PPV-based luminescent liquid-crystalline polymer, thiophene-based luminescent polymer, (thiophene-fluorene)-based luminescent copolymer, alkylthiophene-based luminescent copolymer, ethylene oxide oligomer-added PPV-based luminescent polymer, (carbazoyl methacrylate-coumarin)-based luminescent copolymer, n-type wholly aromatic oxadiazole-based luminescent polymer, carbazoyl cyanoterephthalylidene-based luminescent polymer, heat-resistant, radiation-resistant naphthalimide-based luminescent polymer, aluminum chelate-based luminescent polymer, and octafluorobiphenyl-group-containing luminescent polymer.

Examples of the σ-conjugated polymer material include polysilane-based luminescent polymer.

Examples of the low-molecular dye-containing polymer-based material include carbazole side chain-coupled PMMA-based luminescent polymer and polysilane/dye-based luminescent composition.

Examples of the dopant include Eu complex-doped phosphorescence emitting material, triallylpyrazoline dopant compound, coronene-doped PVK luminescent material, thiophene-based compound-doped (PVK/PBD) luminescent material, Ir complex-doped PVK-based luminescent material, dipyrazole pyridine-based compound-doped luminescent material, pyran-based compound-doped Alq3 luminescent material, reduced porphyrin-doped Alq3 luminescent material, coumarin- or quinacridone-doped Alq-based luminescent material, ammonium salt-doped PVCz-based luminescent polymer, bithiophene-based compound-doped benzimidazole-based luminescent material, (butadiene-based compound: TPA) Co-doped PVK-based luminescent material, dye (TTP: DCM) Co-doped Alq3 luminescent material, ionic luminescent dye-doped PVK-based luminescent material, and dye-doped EL element.

(2) Substrate

It is preferable that the reaction area 2 be formed singularly or plurally in a reaction container (e.g. substrate 6) of a microchip for nucleic acid amplification reaction or the like for example. The reaction container includes at least the reaction area 2 and the reflective component 20 (reflective surface 201), and it is preferable that the reaction container include the sidewall part 21 (sidewall 22) and the phosphor component 23 according to need. In this case, it is preferable that the respective components be disposed around each reaction area 2 in the above-described order, i.e. in the order of the sidewall part 21 (sidewall 22), the phosphor component 23, and the reflective component 20 (reflective surface 201), from the side of the reaction area 2 (see FIGS. 2A to 4C).

(2-a) Method for Manufacturing Substrate

The method for forming the nucleic acid amplification reaction microchip (substrate 6) including the reaction area 2 and the reflective component 20 is not particularly limited.

It is preferable to form the reaction area 2 in the substrate e.g. by wet etching or dry etching of a glass substrate, layer or nanoimprint, injection forming, or cut processing of a plastic substrate layer. The formed reaction area 2 may be filled with reagents for a nucleic, acid amplification reaction in advance.

It is preferable to form the reflective component 20 in the substrate 6 e.g. by forming an inclined surface around the reaction area 2 and depositing a metal film on this surface by sputtering.

The material of the substrate 6 is not particularly limited and it is preferable to accordingly select the material in consideration of the detecting method, the processing easiness, the endurance, and so forth. As this material, a material having optical transparency can be arbitrarily selected depending on the desired detecting method. Examples of the material include glass and various kinds of plastic (polypropylene, polycarbonate, cycloolefin polymer, polydimethylsiloxane (PDMS), etc.).

The method for manufacturing the substrate 6 (micro flow path chip) of the embodiment of the present disclosure will be described in detail below based on the following procedures (A) to (G) (process flow). These procedures are one example of the method for fabricating the substrate 6 of the embodiment of the present disclosure and the manufacturing method is not limited thereto.

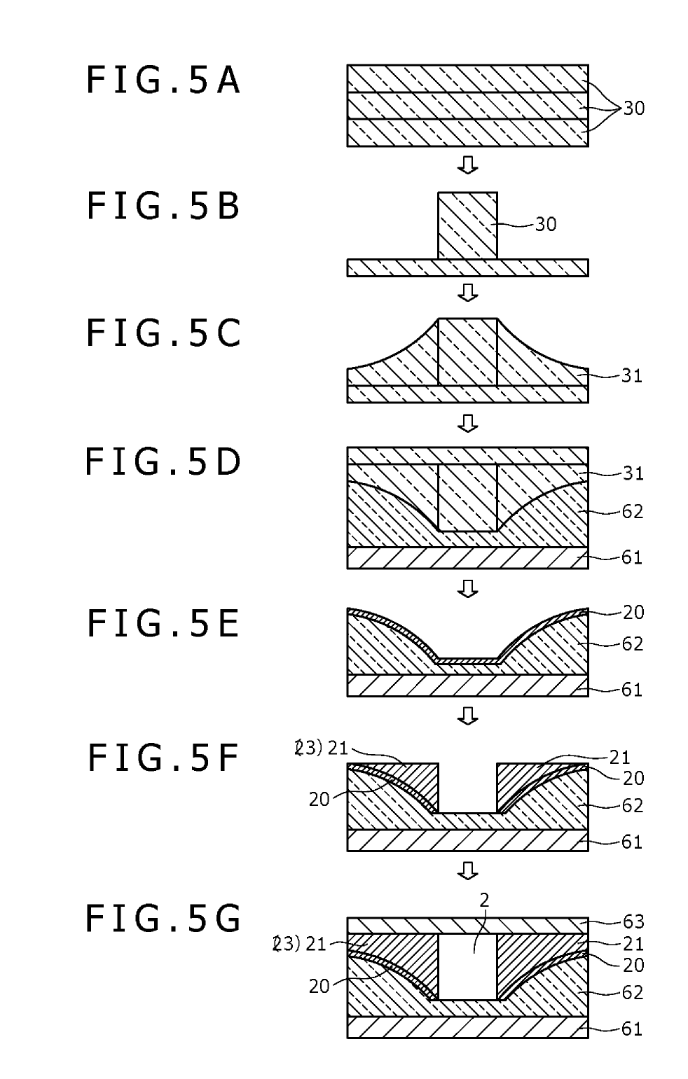

(A) First, a transparent resin 30 (e.g. SU8 photosensitive resin) for forming the reflective component, serving as the mold of the micro flow path chip (substrate 6), is used (see FIG. 5A).

(B) A cylindrical structure to provide a well is fabricated into any shape by photolithography with use of the transparent resin 30 (see FIG. 5B).

(C) Thereafter, a transparent resin having an inclined surface is formed (see FIG. 5C). The transparent resin (resin mold 31) like that shown in FIG. 5C is used as the mold of the substrate 6 (reaction area 2).

A mixture solution of a transparent resin 62 (e.g. PDMS) is cast and cured on a substrate 61 (e.g. glass plate) based on the resin mold 31, and the mold is released by separation (see FIG. 5D).

(D) It is confirmed that a via serving as the well and an inclined surface on the circumference of the via are formed on the transparent resin 62 (substrate 61) from which the mold is released. Thereafter, the reflective component 20 (e.g. metal film: Ag film and subsequently Au film) is formed over the whole surface of the substrate 61 by e.g. sputtering (see FIG. 5E). At this time, it is preferable to use a substance having extremely high reflectance to light of the emission wavelength, e.g. Ag or a metal composed mainly of Ag, as the material of the reflective component 20 (film). This allows end surface emitted light and circulated light returned through reflection by the glass/PDMS to be efficiently reflected by this reflective film, and the light is easily extracted to the outside finally.

(E) A resist pattern having a predetermined circular shape is formed on the reflective component 20 by lithography and the reflective component 20 (metal film) is etched with use of this resist pattern as the mask (see FIG. 5E). Thereby, the reflective component 20 (circular reflective film having an Ag/Au structure) is formed on the inclined surface over the substrate 61 (transparent resin).

(F) Subsequently, by the above-described method, a mixture solution of a transparent resin is cast and cured based on a resin mold for well fabrication like that shown in FIG. 5B and the mold is released by separation, according to the above-described (D) (see FIG. 5F). Thereby, the sidewall part 21 is formed. At this time, the phosphor component 23 (sidewall) may be formed by applying a resin with which a phosphor material is mixed to the sidewall of the sidewall part 21 (not shown). Alternatively, the whole of the sidewall part 21 may be formed as the phosphor component by mixing a phosphor material into the above-described mixture solution cast in the forming of the well shape.

(G) A substrate 63 (e.g. glass or plastic) is so disposed that the space serving as the reaction area 2 is formed.

Through the above-described procedures, the micro flow path chip (substrate 6) having the reaction area 2 according to the embodiment of the present disclosure is obtained.

A substrate having a reflective component for reflection into the light incident surface direction (see e.g. FIG. 2B) can be Obtained by fabricating the substrate according to the above-described method and turning over the substrate after its completion for example.

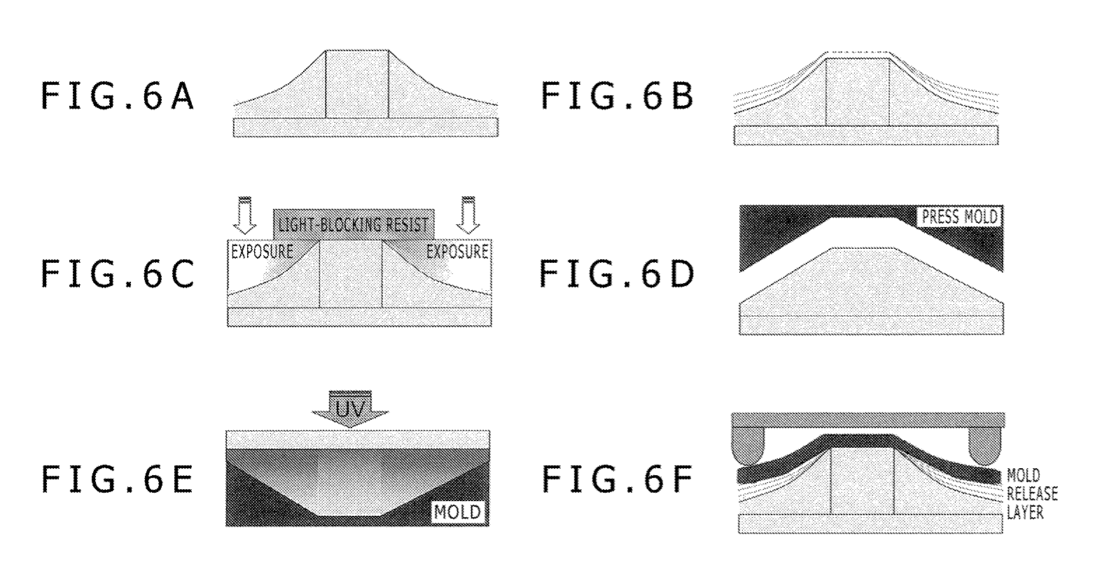

Examples of the method for manufacturing the above-described resin mold 31 include, but not particularly limited to, the following methods (see FIGS. 6A to 6F).

In a first method (see FIG. 6A), the inclined surface is automatically set to an angle of θ₂ by applying a transparent resin over the whole surface by spin-coating.

In a second method (see FIG. 6B), the inclined surface is set to the angle of θ₂ by applying a transparent resin by e.g. spin-coating and then curing and shrinking this transparent resin.

In a third method (see FIG. 6C), a transparent resin is formed by a photolithography technique. Specifically, the inclined surface is set to the angle of θ₂ by using a resist (photosensitive resin) as the transparent resin and performing application, exposure, and development of this resist.

In a fourth method (see FIG. 6D), the inclined surface is set to the angle of θ₂ by press molding of a transparent resin with use of a predetermined mold.

In a fifth method (see FIG. 6E), the inclined surface is set to the angle of θ₂ by thermal imprint of a transparent resin.

In a sixth method (see FIG. 6E), the inclined surface is set to the angle of θ₂ by UV imprint molding of a transparent resin.

In a seventh method (see FIG. 6F), the inclined surface is set to the angle of θ₂ by applying a transparent resin by e.g. spin-coating and then curing this transparent resin while this transparent resin is pressed against an elastically-deformable mold release layer.

(3) Nucleic Acid Amplification Reaction

In the embodiments of the present disclosure; “nucleic acid amplification reaction” includes existing polymerase chain reaction (PCR) in which a temperature cycle is implemented and various kinds of isothermal amplification methods involving no temperature cycle. Examples of the isothermal amplification method include loop-mediated isothermal amplification (LAMP) method, smart amplification process (SMAP) method, nucleic acid sequence-based amplification (NASBA) method, isothermal and chimeric primer-initiated amplification of nucleic acids (ICAN) method (registered trademark), transcription-reverse transcription concerted (TRC) method, strand displacement amplification (SDA) method, transcription-mediated amplification (TMA) method, and rolling circle amplification (RCA) method.

In addition, “nucleic acid amplification reaction” widely encompasses nucleic acid amplification reactions based on a temperature-varying or isothermal process for the purpose of amplification of a nucleic acid. Furthermore, these nucleic acid amplification reactions encompass also reactions accompanied by quantification of the amplified nucleic acid strand, such as real-time PCR (RT-PCR) method and RT-LAMP method.

“Reagent” includes reagents used to obtain an amplified nucleic acid strand in the above-described nucleic acid amplification reaction, specifically oligonucleotide primer with a base sequence that is complementary with the target nucleic acid strand, nucleic acid monomer (dNTP), enzyme, and reaction buffer solution (buffer) solute.

In the above-described PCR method, an amplification cycle of “thermal denaturation (about 95° C.)→primer annealing (about 55 to 60′C.)→extension reaction (about 72° C.)” is continuously carried out.

The above-described LAMP method is a method in which dsDNA is obtained as the amplification product from DNA and RNA at a constant temperature by utilizing loop forming of the DNA. As one example, the following components (i), (ii), and (iii) are added and the process proceeds through incubation at a temperature at which the inner primer can form base paring that is stable for the complementary sequence on the template nucleic acid and the strand-displacing polymerase can keep the enzyme activity. It is preferable that the incubation temperature be 50 to 70° C. and the time be about one minute to 10 hours.

component (i) two kinds of inner primers, or further two kinds of outer primers, or further two kinds of loop primers; component (ii) strand-displacing polymerase; component (iii) substrate nucleotide

(3-a) Method for Detecting Nucleic Acid Amplification (Product)

Examples of the method for detecting the above-described nucleic acid amplification include a method of using a turbidity substance and a method of using a fluorescent substance or chemiluminescence substance.

Examples of the method of using a turbidity substance include a method of using a precipitated substance generated due to the pyrophosphoric acid resulting from the nucleic acid amplification reaction and a metal ion that can be coupled to it. This metal ion is a monovalent or divalent metal ion. When being coupled to the pyrophosphoric acid, it forms a salt that is insoluble or poorly-soluble in water and becomes the turbidity substance.

Specific examples of the metal ion include alkali metal ion, alkaline earth metal ion, and divalent transition metal ion. Among them, one or more kinds of metal ions selected from alkaline earth metal ions such as magnesium (II), calcium (II), and barium (II); and divalent transition metal ions such as zinc (II), lead (II), manganese (II), nickel (II), and iron (II) are preferable. Magnesium (II), manganese (II), nickel (II), and iron (II) are particularly preferable.

It is preferable that the concentration of the added metal ion be in the range of 0.01 to 100 mM. It is preferable to set the detection wavelength to 300 to 800 nm.

Examples of the method of using a fluorescent substance or a chemiluminescence substance include an intercalate method of using a fluorescent dye (derivative) that is specifically intercalated into a double-stranded nucleic acid and emits fluorescence, and a labeled probe method of using a probe obtained by coupling a fluorescent dye to oligonucleotide that is specific to the nucleic acid sequence to be amplified.

Examples of the labeled probe method include hybridization (Hyb) probe method and hydrolysis (TaqMan) probe method.

The Hyb probe method is a method of using two kinds of probes, i.e. a probe labeled with a donor dye that is so designed that two kinds of probes get close to each other in advance, and a probe labeled with an acceptor dye. When these two kinds of probes are hybridized with the target nucleic acid, the acceptor dye excited by the donor dye emits fluorescence.

The TaqMan probe method is a method of using a probe that is so labeled that a reporter dye and a quencher dye get close to each other. This probe is hydrolyzed in nucleic acid extension. At this time, the quencher dye and the reporter dye get separated, and fluorescence is emitted in response to excitation of the reporter dye.

Examples of the fluorescent dye (derivative) used in the method of using a fluorescent substance include SYBR (registered trademark) Green I, SYBR (registered trademark) Green II, SYBR (registered trademark) Gold, YO (Oxazole Yellow), TO (Thiazole Orange), PG (Pico (registered trademark) Green), and ethidium bromide.

Examples of the organic compound used in the method of using a chemiluminescence substance include luminol, lophine, lucigenin, and oxalate.

(4) Irradiating Unit

The irradiating unit 3 may be any unit as long as it includes a light source 3a and has such a configuration that light L1 emitted from the light source is irradiated to the reaction area 2. For example, the light source 3a supported by a support body 3b may be disposed above and/or below the reaction area 2 (see FIG. 1). Furthermore, for example, an optical guide component to guide the light L1 emitted from the light source 3a to the reaction area 2 may be disposed (not shown).

It is preferable that the irradiating unit 3 include the optical guide component. A light incident end part is made in the optical guide component and light emitted from one or plural light sources 3a is incident on the light incident end part. Components (e.g. prism, reflective plate, and concave and convex part) for guiding the incident light L to the respective reaction areas are provided inside the optical guide component.

By disposing the optical guide component, the number of light sources can be reduced and uniform light can be irradiated to one or plural reaction areas 2 on the substrate 6. Furthermore, the detection sensitivity and the detection accuracy in turbidity detection are also favorable. In addition, due to the reduction in the number of light sources, size reduction of the whole device, particularly thickness reduction, is also permitted and power consumption reduction is also enabled.

Although the light source 3a is not particularly limited, a light source that emits the desired light allowing favorable detection of the target nucleic acid amplification product is preferable as the light source 3a. Examples of the light source 3a include laser light source, white or single-color light emitting diode (LED), mercury lamp, and tungsten lamp. Among them, the LED is preferable because it allows power consumption reduction and cost reduction. Furthermore, the LED is advantageous because it also enables achievement of the desired light component if various kinds of filters are used.

The laser light source is not particularly limited by the kind of laser light. A light source that emits e.g. argon (Ar) ion laser, helium-neon (He—Ne) laser, dye laser, or krypton (Kr) laser is enough as the laser light source. As this laser light source, one kind of laser light source may be used or two or more kinds of laser light sources may be used in combination freely.

(5) Temperature Control Unit

The temperature control unit 4 is to heat the reaction area 2. Examples of the temperature control unit 4 include, but not particularly limited to, heater of a Peltier element or the like and ITO heater having optical transparency.

Examples of the shape of the temperature control unit 4 include thin film shape and flat plate shape.

It is preferable that the temperature control unit 4 be disposed at such a position that heat is easily transferred to the reaction area 2. For example, it is preferable that the temperature control unit 4 be disposed close to the reaction area 2. Specifically, it may be disposed at any of positions such as positions above, below, and beside the reaction area 2 and a position at the outer circumference of the reaction area 2.

Particularly, it is preferable that the temperature control unit 4 have a thin film or flat plate shape and be disposed above and/or below the reaction area 2. In this case, the temperature control unit 4 may be disposed as a substrate support mounting. Furthermore, a hole may be made in the temperature control unit 4 so that light may pass through it. This eliminates the need to increase the distance from the heat source and thus facilitates temperature control inside the reaction area 2. Therefore, the detection sensitivity and the detection accuracy are enhanced.

(6) Light Detecting Unit

The light detecting unit 5 may be any unit as long as it is such a mechanism as to be capable of detecting the amount of light of light beams L3 and L4 (L5) obtained by reflecting the side light from the reaction area 2 by the reflective component 20. The light detecting unit 5 is provided with at least an optical detector 5a and this optical detector 5a is accordingly supported by a support body 5b. It is enough that each optical detector 5a is so disposed as to correspond to guided light and the optical detectors 5a are disposed one-dimensionally, two-dimensionally, or three-dimensionally for example.

Examples of the optical detector 5a include, but not limited to, area imaging elements such as photodiode (PD) array, CCD (Charge Coupled Device) image sensor, and CMOS (Complementary Metal Oxide Semiconductor) image sensor, small optical sensor, line sensor scan, and photomultiplier tube (PMT). Any of them may be combined arbitrarily. A fluorescent substance, a turbidity substance, or the like generated by a nucleic acid amplification reaction is detected by the optical detector 5a.

An excitation filter and a fluorescent filter may be disposed in the nucleic acid amplification reaction device 1 of the embodiment of the present disclosure arbitrarily. By the excitation filter, a light component having the desired specific wavelength can be obtained depending on the method for detecting a nucleic acid amplification reaction and an unnecessary light component can be removed. By the fluorescent filter, light is turned to the light component (scattered light, transmitted light, and fluorescence) necessary for detection. This enhances the detection sensitivity and the detection accuracy.

### 2. Operation of Nucleic Acid Amplification Reaction Device 1

The operation of the above-described nucleic acid amplification reaction device 1 and a nucleic acid amplification reaction method by use of it will be described below.

(1) Detection of Light Component Derived from Turbidity Substance in Nucleic Acid Amplification Reaction

With reference to FIG. 1 and FIG. 2A, a description will be made below about a nucleic acid amplification reaction method of detecting the amount of scattered light (the amount of fluorescence) due to a turbidity substance formed from the pyrophosphoric acid and a metal salt.

(1-a) When Phosphor Component 23 is not Present in Substrate 6

<1aA step> The light L1 is emitted from the light source 3a and becomes light L2 (excited light) due to the excitation filter 8. This light L2 is irradiated to the reaction area 2 serving as the reaction filed of an amplification reaction of a nucleic acid.

<1aB step> At this time, a substance precipitated in the nucleic acid amplification reaction (turbidity substance) is generated and thereby the degree of light scattering increases. The light L2 is irradiated to the precipitated substance, generated in association with the progression of the nucleic acid amplification reaction in the reaction area 2. At this time, the scattered light L3 and the side scattered light L4 are generated from the precipitated substance in the reaction area 2.

<1aC step> The side scattered light L4 is reflected by the reflective component 20 (reflective surface 201) disposed on the lateral side of the reaction area 2 and output in the light output surface direction.

<1aD step> Regarding the output light L4, the amount of light is detected by the light detecting unit 5 (optical detector 5a). That is, the amount of scattered light due to the precipitated substance generated in association with the progression of the amplification reaction is detected.

It is also possible to detect the scattered light L3 by light detecting unit (not shown) such as a CCD. However, it is also possible to prevent the passage of the scattered light L3 by disposing a light-blocking substance in the substrate 6.

(1-b) When Phosphor Component 23 is Present in Substrate 6

<1bA step> This step is the same as the above-described <1aA step>.

<1bB step> This step is the same as the above-described <1aB step>.

<1bC step> The side scattered light L4 from the reaction area 2 is transmitted through the phosphor component 23 (sidewall including a phosphor component, phosphor component layer, or the like) to thereby become fluorescence (light L5). This light L5 is reflected by the reflective component 20 (reflective surface 201) disposed on the lateral side of the reaction area 2 and output in the light output surface direction.

<1bD step> The output light L5 is detected by the light detecting unit 5 (optical detector 5a) as the amount of light. That is, the precipitated substance generated in association with the progression of the amplification reaction is detected based on the amount of fluorescence.

The scattered light L3 is the same as that in the above-described case (1-a).

(2) Detection of Light Component Derived from Fluorescent Substance in Nucleic Acid Amplification Reaction

With reference to FIG. 1 and FIG. 2A, a description will be made below about a nucleic acid amplification reaction method of detecting a fluorescent substance generated in a nucleic acid amplification reaction.

(2-a) When Phosphor Component 23 is not Present in Substrate 6

<2aA step> This step is the same as the above-described <1aA step>.

<2aB step> The light L2 is irradiated to a fluorescent substance generated in association with the progression of a nucleic acid amplification reaction in the reaction area 2. At this time, the amount of fluorescence increases due to the generation of the fluorescent substance in the nucleic acid amplification reaction. Accordingly, the forward fluorescence L3 and the side fluorescence L4 are generated from the fluorescent substance in the reaction area 2.

<2aC step> The light L4 is reflected by the reflective component 20 (reflective surface 201) disposed on the lateral side of the reaction area 2 and output in the light output surface direction.

<2aD step> The output light L4 is detected by the light detecting unit 5 (optical detector 5a) as the amount of light. That is, the amount of fluorescence due to the fluorescent substance generated in association with the progression of the amplification reaction is detected.

It is also possible to detect the fluorescence L3 by another light detecting unit. However, it is also possible to prevent the passage of the fluorescence L3 by disposing a light-blocking substance in the substrate 6.

(2-b) When Phosphor Component 23 is Present in Substrate 6

<2bA step> This step is the same as the above-described <1aA step>.

<2bB step> The light L4 from the reaction area 2 is transmitted through the phosphor component 23 (sidewall including a phosphor component, phosphor component layer, or the like) to thereby become a fluorescence component having a specific wavelength (light L5). This light L5 is reflected by the reflective component 20 (reflective surface 201) disposed on the lateral side of the reaction area 2 and output in the light output surface direction.

<2bC step> Regarding the light L5, the amount of output light is detected by the light detecting unit 5 (optical detector 5a). That is, the fluorescent substance generated in association with the progression of the amplification reaction is detected based on the amount of fluorescence of the light component having the specific wavelength.

The fluorescence L3 is the same as that in the above-described case (2-a).

### 3. Nucleic Acid Amplification Reaction Device

### Second Embodiment

FIG. 7 is a schematic conceptual diagram schematically showing a nucleic acid amplification reaction device 1 according to a second embodiment of the present disclosure. Description of the same configuration as that in the first embodiment is omitted.

The nucleic acid amplification reaction device 1 according to the embodiment of the present disclosure (second embodiment) includes at least the detachable substrate 6 having the reaction area 2 and the reflective component 20, the irradiating unit 3, and the light detecting unit 5, and may include the temperature control unit 4 arbitrarily.

In the nucleic acid amplification reaction device 1 of the embodiment of the present disclosure, the light detecting unit 5 is disposed between the irradiating unit 3 and the reaction area 2 (substrate 6).

The excitation filter 8 and the collecting lens 9 may be disposed between the light detecting unit 5 and the irradiating unit 3 arbitrarily. Furthermore, the collecting lens 11 and the fluorescent filter 10 may be disposed between the light detecting unit 5 and the reaction area 2 arbitrarily.

According to need, a light detecting unit 51 may be disposed on the light output surface side of the reaction area 2 and a fluorescent filter (not shown) may be provided between the reaction area 2 and the light detecting unit 51. This allows detection for initialization of the initial value of irradiated light and enhances the detection sensitivity, particularly the detection sensitivity from the timing of the reaction start. A substrate support mounting (temperature control unit 4) may be disposed on the light incident surface side of the reaction area 2 arbitrarily.

### 4. Operation of Nucleic Acid Amplification Reaction Device

### Second Embodiment

The substrate 6 shown in FIG. 2B is preferable as the substrate 6 (micro flow path chip) mounted in the above-described nucleic acid amplification reaction device 1 (second embodiment). The side light from the reaction area 2 is reflected by the reflective component 20 (reflective surface 201) to thereby be output in the light incident surface direction by return.

This will be described in detail below together with the operation of the nucleic acid amplification reaction device 1 (second embodiment).

The light L1 from the irradiating unit 3 is transmitted through the excitation filter 8 and becomes the light L2. This light L2 is transmitted through the support body 5b supporting, the light detecting unit 5 and further transmitted through the temperature control unit 4 (substrate support mounting) to be irradiated to the reaction area 2. The light L2 is irradiated to the nucleic acid amplification product in the reaction area 2 and the light L4 generated toward the lateral side is reflected by the reflective component 20 (reflective surface 201) into the light incident surface direction. This reflected light L4 passes through the temperature control unit 4 and goes through the collecting lens 11 and then the fluorescent filter 10, so that the light component is detected by the light detecting unit 5.

### 5. Modification Examples

In the nucleic acid amplification reaction device of the embodiment of the present disclosure, the reaction area 2 after the reaction end can be set on e.g. the temperature control unit 4 and used also as a nucleic acid amplification detecting device.

Furthermore, it is also possible to mount the substrate 6 (micro flow path chip) of the embodiment of the present disclosure in a LAMP device and a PCR device and quantify a nucleic acid with use, of a fluorescent substance or a turbidity substance in the reaction area as an index. The operation of these devices when a turbidity substance is used as an index will be described below.

(1) Operation of RT-LAMP Device

The method for detecting a nucleic acid by a procedure of a step S11 in an RT-LAMP device will be described below.

In a temperature control step (step S11), the temperature is so set that a constant temperature (60 to 65° C.) is kept in the reaction area 2, and thereby a nucleic acid in each reaction area 2 is amplified. In this LAMP method, thermal denaturation from a single-stranded nucleic acid to a double-stranded nucleic acid is unnecessary and primer annealing and nucleic acid extension are repeatedly performed under this isothermal condition.

As a result of this nucleic acid amplification reaction, the pyrophosphoric acid is generated and a metal ion is coupled to this pyrophosphoric acid, so that an insoluble or poorly-soluble salt is formed and this salt acts as a turbidity substance (measurement wavelength 300 to 800 nm). The incident light (light L) is irradiated to this turbidity substance to thereby become scattered light (light L1, L2). The amount of scattered light is measured by the light detecting unit 5 in real time to be quantified. Quantification from the amount of transmitted light is also possible. If the phosphor component 23 is present in the substrate, quantification from the amount of fluorescence is possible.

When a fluorescent substance is used in a nucleic acid amplification reaction, if the substrate includes the phosphor component 23, light can be turned to a specific fluorescence component and quantification from the amount of fluorescence of this specific fluorescence component is permitted.

(2) Operation of RT-PCR Device

The method for detecting a nucleic acid by procedures of a step Sp1 (thermal denaturation), a step Sp2 (primer annealing), and a step Sp3 (DNA extension) in an RT-PCR device will be described below.

In the thermal denaturation step (step Sp1), the temperature is so controlled by the temperature control unit that 95° C. is kept in the reaction area 2 and a double-stranded DNA is turned to a single-stranded DNA through denaturation.

In the subsequent annealing step (step Sp2), the temperature is so set that 55° C. is kept in the reaction area 2. Thereby, the primer is coupled to the base sequence that is complementary with this single-stranded DNA.

In the next DNA extension step (step Sp3), the temperature is so controlled that 72° C. is kept in the reaction area 2. Thereby, by use of the primer as the start point of DNA synthesis, the polymerase reaction is progressed to extend the cDNA.

By repeating the temperature cycle of these steps Sp1 to Sp3, the DNA in each reaction area 2 is amplified. As a result of this nucleic acid amplification reaction, the pyrophosphoric acid is generated and a turbidity substance is detected in the above-described manner, so that the amount of nucleic acid is quantified as described above. If a phosphor component is present in the substrate, quantification from the amount of fluorescence is possible.

When a fluorescent substance is used in a nucleic acid amplification reaction, if the substrate includes the phosphor component 23, light can be turned to a specific fluorescence component and quantification from the amount of fluorescence of this specific fluorescence component is permitted.

It is preferable that, in the nucleic acid amplification reaction method of the embodiment of the present disclosure, the side light that is generated due to light irradiation and is from the reaction area serving as the reaction field of a nucleic acid amplification reaction be guided into the light output surface direction and/or the light incident surface direction by the reflective component disposed around this reaction area and the amount of guided light be detected by the light detector. Furthermore, it is preferable that the side light be side scattered light and the amount of fluorescence arising from transmission of this side scattered light through a phosphor component be detected. This makes it possible to easily extract scattered light and fluorescence accordingly. Thus, nucleic acid amplification can be measured easily with higher sensitivity. In addition, the need to use an expensive organic fluorescent probe is eliminated. Therefore, measurement at low cost is permitted and quality retention of reagents is enhanced. Moreover, there is an advantage that using the method causes a trouble neither in reaction detection by the related-art turbidity detection nor in optical detection by an organic fluorescent probe.

### Working Example

### Manufacturing Example 1

### Fabrication of Microchip with Reflective Mirror

First, a cylindrical structure to provide a well was fabricated into any shape by photolithography with use of an SU8 photosensitive resin serving as the mold of a micro flow path chip.

The inclined surface was automatically set to an angle of θ2 by applying a transparent resin over the whole surface by spin-coating.

A mixture solution of PDMS was cast and cured based on the above-described fabricated mold, and the mold was released by separation.

It was confirmed that a via serving as the well and an inclined surface on the circumference of the via were formed on the PDMS resin from which the mold was released. Thereafter, Ag film and Au film were sequentially formed over the whole surface of the PDMS substrate by e.g. sputtering. Furthermore, a resist pattern having a predetermined circular shape was formed thereon by lithography and the Ag film and Au film were etched with use of this resist pattern as the mask. Thereby, a circular reflective film having an Ag/Au structure was formed on the transparent resin. As the material of the reflective film, a substance having extremely high reflectance to light of the emission wavelength, e.g. Ag or a metal composed mainly of Ag, was used. This is because this allows end surface emitted light and circulated light returned through reflection by the glass/PDMS to be efficiently reflected by this reflective film and the light is easily extracted to the outside finally.

### Test Example 1

### Test Method

(1) Immobilization Drying of Single-Stranded DNA Primer Reagent

A primer solution for LAMP was mixed.

The design of the LAMP method primer was carried out by utilizing six domains, i.e. F3 domain, domain, F1 domain, B1 domain, B2 domain, and B3 domain, from the 5′ side of the target sequence. In the basic LAMP method, four kinds of primers (two kinds of inner primers and two kinds of outer primers) are used. The inner primers couple F1c to F2 and couple B1c to B2. The forward loop primer was set for the complementary strand for the domain between F1 domain and F2 domain, and the backward loop primer was set for the complementary strand for the domain between B1 domain and B2 domain.

The free energy of the 3′ end of F2/B2, F3/B3, and LF/LB and the 5′ end of F1c/B1c was set equal to or lower than −4 kcal/mol.

FIP-BIP and F3 and B3 domains were designed from the whole target domain and subsequently a primer set obtained by selecting and combining one pair of F3 and B3 domains was designed for each FIP-BIP domain. The combination of FIP-BIP and F3 and B3 domains starts from the 5′ end and continues to the 3′ end. Subsequently, the combination starts from the 5′ end again and the design is progressed toward the 3′ end, and at most three kinds of F3-B3 are combined for one FIP-BIP. The codes of the primer designed by Primer Explorer are shown in the following Table 1.

TABLE 1

| | |
|---|---|
| Sequence Number 1 | FIP (Forward Inner Primer): |
| | TACACCTTTGTTCGAGTCATGATGAAAGGTTTGAGATATTCCCA |
| Sequence Number 2 | BIP (Backward Inner Primer): |
| | CTCATGCTGGAGCAAAAAGCTTCATTTGCTGAGCTTTGGGT |
| Sequence Number 3 | F3: |
| | GCAATTGAGCTCAGTGTCAT |
| Sequence Number 4 | B3: |
| | TCTTTCCCTTTATCATTAATGTAGG |
| Sequence Number 5 | LF: |
| | TGGGCCATGAACTTGTCT |
| Sequence Number 6 | LB: |
| | GGCTAGTTAAAAAAGGAAATTCA |

(2) Chip Bonding Fabrication

The PDMS substrate in which enzyme and primer were immobilized to all wells was subjected to DP ashing under a condition of O₂: 10 cc, 100 W, and 30 seconds to turn the surface to a hydrophilic surface. Then the PDMS substrate was bonded to a cover glass in vacuum.

(3) LAMP Reaction

The PDMS was penetrated by a painless needle and an extraction mixture solution for reaction with a quantified copy number was introduced into the flow path in the chip. Next, the chip was set in a fluorescence detecting device including a fluorescence detector and a heater over a measurement substrate for each reaction area (well). In this device, excitation light from an LED was irradiated from above each well in the microchip substrate simultaneously with the reaction and light scattered by a reaction by-product in the reaction area was detected.

The excitation light scattered in the well was irradiated to an inorganic phosphor of the well sidewall and fluorescence was emitted.

This fluorescence was detected and measured by a fluorescence detecting photodetector provided below the microchip substrate reaction area disposed on the optical axis of the excitation light source.

(4) Result Determination (about Heating Time)

According to the result of measurement at every 0.1 minutes after the LAMP reaction start and the result of measurement at shorter intervals, the product was obtained and the fluorescence intensity started to become high in about nine minutes in an influenza virus system. However, it was not until the elapse of 16 minutes after the reaction start that white turbidity in the well could be visually confirmed.

In the related-art turbidity system utilizing transmitted light, the S/N ratio is low and the determination is difficult until the particular size of the magnesium pyrophosphate colloid becomes sufficiently large and white turbidity is caused.

In contrast, the projected surface area was increased by extracting laterally scattered light. Thus, a sufficiently high S/N ratio could be ensured because almost no side scattering is present in a transmissive optical system including no scattering object.

Furthermore, as an advantage of the reflective type, it could be confirmed that the S/N ratio with respect to incident light could be enhanced because a light receiver could be disposed on the incident light side and a filter could be applied to the light receiver if fluorescence was obtained by side scattered light.

The nucleic acid amplification reaction device according to the embodiments of the present disclosure can ensure a sufficiently-high. S/N ratio by reflecting the side light by the reflective component, and allows measurement with high detection sensitivity although it is easy to use. In addition, by using the phosphor component, removal of an unnecessary light component and achievement of a specific light component are also permitted. Thus, despite low cost, easy measurement with high detection sensitivity is possible.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

The application is claimed as follows:

1. A nucleic acid amplification reaction device comprising:
a reaction area configured to serve as a reaction field of a nucleic acid amplification reaction;
an irradiating unit configured to irradiate light to the reaction area; and
a light detecting unit configured to detect the amount of reflected light,
wherein a reflective component that reflects side light generated in the reaction area due to light irradiation from the irradiating unit and guides the light to the light detecting unit is disposed.

2. The nucleic acid amplification reaction device according to claim 1, wherein the reflective component is disposed around the reaction area in such a manner as to guide side light from the reaction area into a light output surface direction and a light incident surface direction.

3. The nucleic acid amplification reaction device according to claim 1, wherein the reflective component is disposed around the reaction area in such a manner as to guide side light from the reaction area into a light output surface direction.

4. The nucleic acid amplification reaction device according to claim 1, wherein the reflective component is disposed around the reaction area in such a manner as to guide side light from the reaction area into a light incident surface direction.

5. The nucleic acid amplification reaction device according to claim 1, wherein one or a plurality of phosphor components is provided between the reaction area and the reflective component.

6. A substrate comprising
a reflective component configured to reflect side light from a reaction area serving as a reaction field of a nucleic acid amplification reaction.

7. The substrate according to claim 6, further comprising
a phosphor component configured to be provided between the reaction area and the reflective component.

8. A nucleic acid amplification reaction method comprising:
guiding side light that is generated due to light irradiation and is from a reaction area serving as a reaction field of a nucleic acid amplification reaction into a light output surface direction and a light incident surface direction by a reflective component disposed around the reaction area; and
detecting the amount of guided light by a light detector.

9. The nucleic acid amplification reaction method according to claim 8, wherein the side light is side scattered light and the amount of fluorescence arising from transmission of the side scattered light through a phosphor component is detected.

10. A nucleic acid amplification reaction method comprising:
guiding side light that is generated due to light irradiation and is from a reaction area serving as a reaction field of a nucleic acid amplification reaction into a light output surface direction by a reflective component disposed around the reaction area; and
detecting the amount of guided light by a light detector.

11. The nucleic acid amplification reaction method according to claim 10, wherein the side light is side scattered light and the amount of fluorescence arising from transmission of the side scattered light through a phosphor component is detected.

12. A nucleic acid amplification reaction method comprising:
guiding side light that is generated due to light irradiation and is from a reaction area serving as a reaction field of a nucleic acid amplification reaction into a light incident surface direction by a reflective component disposed around the reaction area; and
detecting the amount of guided light by a light detector.

13. The nucleic acid amplification reaction method according to claim 12, wherein the side light is side scattered light and the amount of fluorescence arising from transmission of the side scattered light through a phosphor component is detected.


SEQUENCE LISTING (6 sequences)

SEQ ID NO: 1 - DNA, 44 bp, Artificial Sequence
  Synthesized  FIP primer
tacacctttgttcgagtcatgatgaaaggtttgagatattccca

SEQ ID NO: 2 - DNA, 41 bp, Artificial Sequence
  Synthesized BIP primer
ctcatgctggagcaaaaagcttcatttgctgagctttgggt

SEQ ID NO: 3 - DNA, 20 bp, Artificial Sequence
  Synthesized F3 primer
gcaattgagctcagtgtcat

SEQ ID NO: 4 - DNA, 25 bp, Artificial Sequence
  Synthesized B3 primer
tctttccctttatcattaatgtagg

SEQ ID NO: 5 - DNA, 18 bp, Artificial Sequence
  Synthesized LF primer
tgggccatgaacttgtct

SEQ ID NO: 6 - DNA, 22 bp, Artificial Sequence
  Synthesized LB primer
ggctagttaaaaaaggaaattc